(19) United States
(12) Patent Application Publication
(10) Pub. No.: US 2012/0143347 A1
(43) Pub. Date: Jun. 7, 2012

(54) Elastomeric, Polymeric Bone Engineering and Regeneration Compositions and Methods of Making

(75) Inventors: Yadong Wang, Allison Park, PA (US); Charles Sfeir, Pittsburgh, PA (US)

(73) Assignee: University of Pittsburgh - Of The Commonwealth System Of Higher Education, Pittsburgh, PA (US)

(21) Appl. No.: 13/310,033

(22) Filed: Dec. 2, 2011

Related U.S. Application Data
(60) Provisional application No. 61/419,523, filed on Dec. 3, 2010.

(51) Int. Cl. A61F 2/28

(52) U.S. Cl. 623/23.61

(57) ABSTRACT

The invention relates to elastomeric, polymeric bone substitute compositions, methods for their preparation and their use in clinical applications, such as bone engineering and regeneration applications. The bone substitute compositions are osteoconductive in the absence of a biomolecule component. The bone substitute compositions are capable to regenerate bone in vitro or in vivo. The bone substitute compositions can be implanted into a bone defective site of a patient, and particularly, in the orthopedic, dental and caraniofacial areas of the patient.

### CROSS-REFERENCE TO RELATED APPLICATIONS

This application claims priority under 35 U.S.C. §119(e) from provisional U.S. patent application No. 61/419,523, entitled “Elastomeric, Polymeric Bone Engineering and Regeneration Compositions and Methods of Making” filed on Dec. 3, 2010, the contents of which are incorporated herein by reference.

### FIELD OF THE INVENTION

The invention relates to elastomeric, polymeric bone substitute compositions, methods for their preparation, and their use in clinical applications, such as bone engineering and regeneration applications. The elastomeric, polymeric bone substitute compositions are particularly useful to regenerate bone in a bone defective site of a patient.

### BACKGROUND OF THE INVENTION

It is known in the art to use biodegradable polymers in various fields of medicine, such as tissue engineering, drug delivery and vivo sensing. Many biomedical devices are implanted in a body of a patient and therefore, biocompatibility of these devices is also important. Further, it is known in the art to construct implantable matrices or scaffolds of biodegradable, biocompatible materials to regenerate bone in a bone defective site of a patient.

Bone is a dynamic tissue wherein the direction and intensity of applied forces can play a pivotal role in the pattern of its remodeling and regeneration. Since an osteoconductive scaffold or construct is primarily inserted in a bone defective site, e.g., into the gap, to be regenerated, the biological and mechanical properties of such material are expected to significantly impact the regeneration process. For example, the biological and mechanical properties of the bone substitute material can affect the number and types of the adherent cells, their migration and proliferation patterns and their differentiation from the stem/progenitor genotype into bone matrix-depositing cells. In addition to genetic influences, the microenvironment, whether biologically through ion concentration and cell-to-cell interaction or mechanically through cell-sensed or perceived stiffness of the scaffold/substrate, influences the differentiation process directing a stem cell into its self renewal cycle or through a state of specific tissue(s) progenitor or an end-differentiation fate.

It has been estimated that approximately 6.2 million bone fractures occur annually in the United States. Further, it has been estimated that 5% to 10% fail to heal properly due to delayed or non-union. Current therapies offer limited regenerative options. The development of effective long-term therapies for bone regeneration, particularly, in the craniofacial area or orthopedic applications, is highly desirable for the novel tissue engineering modality of treatment.

Various polymer- and ceramic-based scaffolds have been studied for use as bone substitute compositions for the purpose of bone regeneration. Many of the bone substitute compositions known in the art do not exhibit elastomeric properties. For example, known materials for scaffolds exhibit a stiffness that is comparable to mature bone. Ceramic materials have been known to be used to in a bone defective site of a patient. These materials have disadvantages associated with them such as brittleness. Thus, there is a desire in the art to develop a bone substitute composition to exhibit elastomeric properties such that the material can be shaped and formed while providing mechanical stability and structural integrity. In addition, it is desirable for the bone substitute composition to exhibit other properties, such as biocompatibility, resorbability and a porous structure, to promote and enhance bone regeneration.

### SUMMARY OF THE INVENTION

In one aspect, the invention provides an elastomeric, polymeric bone substitute composition capable to regenerate bone in the absence of a biomolecule component.

The bone substitute composition can include a condensation polymer formed by a reaction of glycerol and a diacid. The diacid can include sebacic acid. The condensation polymer can include poly(glycerol sebacate).

The bone substitute composition can regenerate bone in the absence of a growth compound.

The bone substitute composition can be seeded with bone-forming cells. In one embodiment, the bone-forming cells are osteoprogenitor cells.

The bone substitute composition can include a porogen.

The bone substitute composition can include a polymer selected from the group consisting of biodegradable polymers, non-bidegradable polymers and mixtures thereof.

The bone substitute composition includes poly(glycerol sebacate) which is capable to regenerate bone in a bone defective site of a patient.

In another aspect, the invention provides a method of preparing a bone substitute composition. The method includes reacting glycerol and a diacid to form a condensation polymer, in the absence of a biomolecule component.

In still another aspect, the present invention provides a method for regenerating bone in a bone defective site of a patient. The method includes preparing a bone substitute composition including a condensation polymer containing glycerol and a diacid, implanting the bone substitute composition in the absence of a biomolecule component, in a bone defective site of a patient.

In yet another aspect, the present invention provides a method for regenerating bone. The method includes preparing a bone substitute composition including a condensation polymer which is a reaction product of glycerol and a diacid and a biomolecule component. The bone substitute composition is effective to regenerate bone in a period of 8 weeks or less.

The term “biomolecule,” as used herein, refers to a molecule (e.g., protein, amino acid, peptide, polynucleotide, nucleotide, carbohydrate, sugar, lipid, nucleoprotein, glycoprotein, lipoprotein, steroid, etc.), whether naturally-occurring or artificially created (e.g., by synthetic or recombinant methods), that is commonly found in cells and tissues in a body of a patient. Specific classes of biomolecules include, but are not limited to, enzymes, receptors, neurotransmitters, hormones, cytokines, cell response modifiers, such as growth factors and chemotactic factors, antibodies, vaccines, haptens, toxins, interferons, ribozymes, anti-sense agents, plasmids, DNA, RNA, and combinations thereof.

The term “biocompatible,” as used herein is intended to describe materials that do not elicit a substantial detrimental response in vivo.

As used herein, “biodegradable” polymers refers to polymers that degrade substantially fully (i.e., to the monomeric species) under physiological or endosomal conditions. The biodegradable polymers and the polymer biodegradation byproducts can be biocompatible. Biodegradable polymers are not necessarily hydrolytically degradable and may require enzymatic action to fully degrade.

As used herein, the term “elastomer” means a macromolecular material that can rapidly return to the approximate shape from which it has been substantially distorted by a weak stress.

The phrase “endosomal conditions,” as used herein, relates to the range of chemical (e.g., pH, ionic strength) and biochemical (e.g., enzyme concentrations) conditions likely to be encountered within endosomal vesicles. For example, most endosomal vesicles have an endosomal pH in the range from about 5.0 to 6.5.

The phrase “physiological conditions,” as used herein, relates to the range of chemical (e.g., pH, ionic strength) and biochemical (e.g., enzyme concentrations) conditions likely to be encountered in the intracellular and extracellular fluids of tissues. For example, most tissues have a physiological pH in the range from about 7.0 to 7.4.

The terms “polynucleotide,” “nucleic acid,” or “oligonucleotide” refer to a polymer of nucleotides. The terms “polynucleotide,” “nucleic acid,” and “oligonucleotide” may be used interchangeably. Typically, a polynucleotide includes at least three nucleotides. DNAs and RNAs are polynucleotides. The polymer may include natural nucleotides, such as adenosine, thymidine, guanosine, cytidine, uridine, deoxyadenosine, deoxythymidine, deoxyguanosine, and deoxycytidine); nucleotide analogs, such as, 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine; chemically modified bases; biologically modified bases, such as methylated bases; intercalated bases; modified sugars, such as 2′-fluororibose, ribose, 2′-deoxyribose, arabinose, and hexose; modified phosphate groups, such as phosphorothioates and 5′-N-phosphoramidite linkages and, mixtures and combinations thereof.

According to the present invention, a “polypeptide,” “peptide,” or “protein” includes a string of at least three amino acids linked together by peptide bonds. The terms “polypeptide,” “peptide,” and “protein” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may be employed. One or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification. Modification of the peptide leads to a more stable peptide (e.g., greater half-life in vivo). These modifications may include cyclization of the peptide and the incorporation of D-amino acids. Any modifications are performed such that they do not substantially interfere with the desired biological activity of the peptide.

The terms “polysaccharide,” “carbohydrate,” or “oligosaccharide” refer to a polymer of sugars. The terms “polysaccharide,” “carbohydrate,” and “oligosaccharide,” may be used interchangeably. Typically, a polysaccharide includes at least three sugars. The polymer may include natural sugars, such as glucose, fructose, galactose, mannose, arabinose, ribose, and xylose, modified sugars, such as 2′-fluororibose, 2′-deoxyribose, and hexose and, mixtures and combinations thereof.

As used herein, the term “tissue” refers to a collection of similar cells combined to perform a specific function and any extracellular matrix surrounding the cells.

### BRIEF DESCRIPTION OF THE DRAWINGS

The invention as set forth in the claims will become more apparent from the following detailed description of certain preferred practices thereof illustrated by way of example only and the accompanying figures, wherein:

FIG. 1A shows a plot of compression modulus for PGS polymer tubes in accordance with an embodiment of the invention.

FIG. 1B shows a plot of tensile modulus for PGS polymer tubes in accordance with an embodiment of the invention.

FIG. 1C shows a plot of tensile modulus in air for PGS polymer sheets in accordance with an embodiment of the invention.

FIG. 2A is a SEM image showing viable cells migrating out of a replated PGS scaffold, 20×, in accordance with an embodiment of the invention.

FIG. 2B is a SEM image showing confluent BMSC on the surface of a PGS scaffold, 1000×, in accordance with an embodiment of the invention.

FIG. 2C is a SEM image showing a BMSC attached to the PGS scaffold, 2000×.

FIG. 2D is an increased magnification of the SEM image in FIG. 2C emphasizing the close integrity of a cell to the PGS scaffold, 10000×, in accordance with an embodiment of the invention.

FIG. 3 shows radiographs and μCT images of an ulna defective at T0 day of surgery, 4 weeks following surgery and 8 weeks following surgery.

FIGS. 4A thru 4D show μCT images of a negative control ulna at 8 weeks following surgery, a PGS sample ulna at 4 weeks following surgery in accordance with an embodiment of the invention, a PGS sample ulna at 8 weeks following surgery in accordance with an embodiment of the invention and an intact ulna, respectively.

FIGS. 4E thru 4H show x-rays of a negative control ulna, a PGS sample ulna at 4 weeks following surgery in accordance with an embodiment of the invention, a PGS sample ulna at 8 weeks following surgery in accordance with an embodiment of the invention and an intact ulna, respectively.

FIG. 4I shows a detailed x-ray of a section in FIGS. 4C and 4G indicating a remodeling cortical shell in accordance with an embodiment of the invention.

FIG. 4J shows an x-ray of an intact radius/ulna cross-section showing the flatness of the ulna compared to the radius.

FIG. 4K is a plot of bone volume quantification/mm³ along a 15 mm segment of the 16 mm bone defect for a 4-week PGS sample ulna and an 8-week PGS sample ulna in accordance with embodiments of the invention as compared to a 8-week negative control sample and an intact ulna.

FIG. 4L is a plot of overall bone density of a regenerated bone by a PGS sample ulna at 8 weeks as compared to an intact ulna.

### DETAILED DESCRIPTION OF THE INVENTION

Elastomeric, polymeric bone substitute compositions, methods for their preparation, and their use in clinical applications, such as bone engineering and bone regeneration applications, are provided. The bone substitute compositions can be used to repair or replace defective native bone tissue. In one embodiment, the bone substitute compositions are used to repair or replace defective native bone tissue in orthopedic, dental, and craniofacial areas of a patient. The bone substitute compositions are effective to regenerate bone.

The bone substitute compositions serve as a scaffold for new bone growth that is perpetuated by native bone and therefore, are considered to be osteoconductive. In one embodiment, the bone substitute compositions are osteoconductive in the absence of a biomolecule, such as a biological growth factor. The bone substitute compositions can be used to construct osteoconductive scaffolds for in vitro or in vivo applications.

In one embodiment, a scaffold constructed of a bone substitute composition in accordance with an embodiment of the invention, is injected or implanted in a bone defective site of a patient and is capable to regenerate bone to fill a gap.

The bone substitute compositions exhibit excellent cell and host tissue biocompatibility. In addition, the bone substitute compositions demonstrate one or more of the following characteristics or properties which are particularly beneficial for bone regeneration.
1) Act as a temporary framework that balances mechanical function with delivery of biofactors, such as cells and/or growth factors. The term “temporary” means that as it degrades, the scaffold provides mechanical support of biofactors while providing a smooth transition from a scaffold-occupied defect to a functional tissue-occupied defect. The cell-scaffold interaction closely mimics the interplay between cell-surface receptors and extracellular matrix (ECM) molecules. This interplay is important in regulating cellular functions including survival, adhesion, proliferation, migration, differentiation, and matrix deposition.
2) Allow sufficient vascular in-growth for bone regeneration as bone forms within a vascularized site. This is primarily a factor of the microstructure of the scaffold including its pore size and structure. The pore size is sufficiently large for hierarchical vessels penetration without compromising its mechanical properties and the structure is such that the pores interconnect to facilitate mass transport.
3) Provide an osteoconductive environment where the osteoprogenitor cells are capable (e.g., “comfortable”) to attach, migrate and differentiate to deposit a bone matrix to be mineralized. Hydroxyapatite (HA) and biphasic phosphates provide a “bone friendly” microenvironment which is comparable to mature bone matrix in terms of compressive properties. HA and/or biphasic phosphates can be combined with various synthetic polymeric materials, such as PLGA/PGA, and/or natural polymeric materials, such as collagen and fibrin. Without intending to be bound by any particular theory, it is believed that such combinations produce a material having improved elasticity.
4) Accommodate a change in mechanical load transmitted from the load-bearing surrounding bone to the regenerating segment to regulate trabecular orientation and cell function. The regulation of the cell function is not only limited to soluble and adhesive factors that bind to cell-surface receptors, but also by mechanical extracellular stimuli. Without intending to be bound by any particular theory, it is believed that cell shape, focal adhesion structures and cytoskeletal tension are closely related to and involved in rigidity sensing which allows the cell to distinguish or “feel” the difference between soft and hard. Integrating these cues with other signals can direct differentiation. Further, ECM elasticity can direct cells lineage differentiation in vitro and the effect can be synergistically enhanced with the combination of the suitable soluble induction factors to simulate available micromolecules in vivo. This load-transmitting property, which requires a scaffold with certain elasticity, is in addition to the mechanical stability property of the scaffold into the defective site which provides an uninterrupted regeneration process.

The bone substitute compositions include a condensation polymer formed by the reaction of glycerol and a diacid. The diacid can be selected from a wide variety of materials known in the art. The diacid may include one or more double bonds, and an aromatic group, an amine, a hydroxyl group, a halogen atom, an aliphatic side chain or any combination thereof. Non-limiting examples of suitable diacids include malonic acid, succinic acid, sebacic acid, glutaric acid, adipic acid, pimelic acid, suberic acid, azelaic acid, terephthalic acid, carboxyphenoxypropane and, mixtures and derivatives thereof.

In a preferred embodiment, the diacid is sebacic acid and the condensation polymer is poly(glycerol sebacate).

The amount of glycerol and diacid can vary. The amount used can depend on the desired properties of the resulting condensation polymer, such as its elasticity and rigidity. In alternate embodiments, the ratio of glycerol to diacid can be between 1 and 1.5. In a preferred embodiment, the ratio of glycerol to diacid is 1.

The reaction can optionally include the presence of a porogen. The porogen can be selected from a wide variety of materials known in the art and includes, but is not limited to, recrystallized organic salt, recrystallized inorganic salt, engineered peptide, natural polymer, a composite of natural and synthetic polymers, synthetic biodegradable polymer, natural extra cellular matrix protein and, mixtures and derivatives thereof. In one embodiment, the porogen is an inorganic salt, such as, but not limited to, potassium chloride, sodium chloride, sodium phosphate, sodium citrate, sodium tartrate, sodium acetate and, mixtures and derivatives thereof. In one embodiment, the porogen is a natural polymer, such as, but not limited to, mannitol, collagen, sucrose, fibrin, gelatin, alginate, chitosan, fibrin-gelatin composite, paraffin, polyol, poly lactic-co-glycolic acid (PLGA) and, mixtures and derivatives thereof. In a preferred embodiment, the porogen is azodicarboimide.

The porogen can be present in varying amounts. It is contemplated that the amount of porogen employed can depend on the selection of diacid as well as other components present in the reaction mixture.

The porosity of the bone substitute compositions can vary. It is typical for the lower limit of a porosity range to be about 300 μm. This limit provides for proper tissue migration, nutrients transport and capillaries formation. In alternate embodiments, in accordance with the invention, the porosity is at least about 75 μm or no greater than about 150 μm or in the range of from about 75 μm to about 150 μm. In these embodiments, it is believed without intending to be bound by any particular theory, that the porosity provides a bone substitute composition its initial mechanical strength and stability for host cells trafficking and matrix deposition.

The reaction can optionally include the presence of an alkali halide salt or a water-soluble salt. Suitable alkali halide salts or water soluble salts include a wide variety known in the art. In one embodiment, sodium chloride is used.

The reaction can optionally include the presence of catalyst. The catalyst can be effective to reduce reaction temperature, shorten reaction time, and increase individual chain length. The catalyst should be biocompatible or easily removed. In a preferred embodiment, the catalyst used is the FDA-approved catalyst stannous octoate (bis(2-ethylhexanoate)tin(II)).

The condensation polymer can have a tensile elastic modulus that varies widely. The desired tensile elastic modulus can depend on the intended use or application of the condensation polymer. In one embodiment, the tensile elastic modulus is about 5 MPa or less. In alternate embodiments, the tensile elastic modulus can be less than about 3 MPa, less than about 1 MPa, less than about 0.5 MPa, less than about 0.3 MPa or less than about 0.1 MPa.

The condensation polymer can have a Young's modulus that varies widely. The Young's modulus can be less than about 3 MPa, less than about 1 MPa, less than about 0.5 MPa, less than about 0.1 MPa or less than about 0.01 MPa.

The tensile strength of the condensation polymer can vary. In alternate embodiments, the tensile strength can be at least about 0.10 MPa or no greater than about 0.16 MPa or in the range of from about 0.10 MPa to about 0.16 MPa. In one embodiment, the tensile strength is about 0.13 MPa.

The compressive strength of the condensation polymer can vary. In alternate embodiments, the compressive strength can be at least about 0.03 MPa or no greater than about 0.05 MPa or in the range of from about 0.03 MPa to about 0.05 MPa. In one embodiment, the compressive strength is about 0.038 MPa.

The tensile and compressive strength of the condensation polymer indicates that the bone substitute compositions in accordance with embodiments of the invention exhibit higher elasticity and lower stiffness as compared to known substitute materials which have comparable porosity, such as, PLGA and Ha/β-TCP materials. The stiffness of the condensation polymer resembles bone marrow more than it resembles cortical or cancellous bone. In one embodiment, the stiffness of the condensation polymer falls within a range of “osteogenesis sensing”, e.g., an elastic modulus in the range of from about 25 to about 40 kPa, which favors cells differentiation into the osteogenic lineage. Without intending to be bound by any particular theory, it is believed to be difficult at best for a bone substitute composition to satisfy both cortical and cancellous mechanical properties because the former is about 20 times stiffer than the latter. The condensation polymers in accordance with embodiments of the invention provide a suitable substrate for cortical and cancellous bone development and regeneration because they provide lower stiffness which corresponds with developing skeletal tissue rather than higher stiffness corresponding to mature bone tissue.

Further, without intending to be bound by any particular theory, it is believed that the elasticity of the bone substitute composition allows external mechanical load to be transduced and stimulates the osteogenic progenitor differentiation. Mechanical stimulation is a factor in osteogenic stimulation which can compensate for the amount of loaded cells for regeneration and bridging a gap in a bone defective site.

The condensation polymer may have a crosslink density that varies widely. In alternate embodiments, the crosslink density is about 40% or less, less than about 30%, less than about 20%, less than about 10%, less than about 5%, less than about 1%, less than about 0.5% or less than about 0.05%. In one embodiment, the crosslink density is from about 1% to less than about 40%.

The condensation polymer can be prepared in accordance with a variety of known methods. In one embodiment, the condensation polymer is prepared by combining equal molar amounts of glycerol and a diacid to form a mixture. The mixture is held at a temperature of about 120° C. in an inert atmosphere at a pressure of about 1 Torr for time period of about 24 hours. Further, the mixture is held at a temperature of about 120° C. and a pressure of about 40 mTorr until the mixture forms a polymer having a predetermined cross-link density. The mixture may be held at 40 mTorr for a period of from about 24 hours to about 48 hours.

In one embodiment, wherein the condensation polymer is poly(glycerol sebacate), it is synthesized by polycondensation of 0.1 mole each of glycerol and sebacic acid at a temperature of 120° C. under argon for a period of 24 hours prior to reducing the pressure from 1 Torr to 40 mTorr over a period of 5 hours. The reaction mixture is kept at a pressure of 40 mTorr and a temperature of 120° C. for a period of 48 hours. The yield is a transparent, almost colorless elastomer that does not swell or dissolve in water.

The step of combining reactants can further include adding a porogen, an alkali halide salt, or a water-soluble salt to the mixture. The polymerized mixture may be soaked in water to leach out the porogen.

The bone substitute compositions and the scaffolds constructed therefrom can also optionally include other additives known in the arts of bone tissue engineering and regeneration. For example, HA and biphasic phosphates, such as biphasic calcium phosphate, can be used. In alternate embodiments, HA particles can be mixed with porogen and added to the condensation polymer reaction mixture or a HA solution can be prepared and the condensation polymer or the scaffold constructed therefrom can be coated with the solution or the scaffold constructed of the condensation polymer can be dipped into a bath of calcium phosphate solution.

The method of preparing the condensation polymer can further include modifying hydroxyl groups on the polymer with one or more of a biomolecule, a hydrophilic group, a hydrophobic group, a non-protein organic group, an acid, a small molecule and a bioactive agent.

The condensation polymer, e.g., poly(glycerol sebacate), can be chemically analyzed using a variety of conventional analysis techniques known in the art. Non-limiting examples of chemical analysis techniques include the following: i) a KBr pellet of newly-prepared poly(glycerol sebacate) can be used for FTIR analysis on a Nicolet Magna-IR 550 Spectrometer; ii) a Perkin-Elmer DSC differential scanning calorimeter can be used for DSC measurements; iii) an elemental analysis on vacuum-dried samples can be performed by QTI Inc.; and iv) the water-in-air contact angle can be measured at room temperature using a sessile drop method and an image analysis of the drop profiled with VCA2000 video contact angle system on slabs of polymer fixed on glass slides.

The results of chemical analysis of the yield can show the percent completion of the polymerization reaction.

The condensation polymer can be a thermoset polymer. In this embodiment, an uncrosslinked prepolymer can be processed into various shapes because it can be melted into liquid and is soluble in common organic solvents, such as 1,3-dioxolane, tetrahydrofuran, ethanol, isopropanol, and N,N-dimethylformamide. For example, a mixture of sodium chloride (NaCl) particles of appropriate size and an anhydrous 1,3-dioxolane solution of the prepolymer is poured into a PTFE mold. One skilled in the art will recognize that other salts besides NaCl may also be employed. The polymer is cured in the mold in a vacuum oven at a temperature of 120° C. and a pressure of 100 mTorr. A porous scaffold is obtained after salt leaching in deionized water. A wide variety of known porogens can be used, such as, but not limited to, those described herein. In one embodiment, the porogen is azodicarboimide, which decomposes into nitrogen, carbon dioxide, and ammonia upon heating. An ionic porogen may be selected such that it is soluble in water and does not interfere with polymerization.

The hydroxyl groups on the condensation polymer, e.g., poly(glycerol sebacate), provide sites to which molecules may attach to modify the bulk or surface properties of the bone substitute composition. For example, tert-butyl, benzyl, or other hydrophobic groups may be added to the material to reduce the degradation rate. Polar organic groups, such as methoxy, facilitate adjustment of the degradation rate and hydrophilicity. In contrast, addition of hydrophilic groups, for example, sugars, at these sites increase the degradation rate.

Acids can also be added to the condensation polymer to modify its properties. For example, molecules with carboxylic or phosphoric acid groups or acidic sugars may be added. Charged groups, such as sulfates and amines, may also be attached to the polymer. For example, a charged amino acid, such as arginine or histidine, may be attached to the polymer to modify the degradation rate.

Groups that are added to the polymer may be added via linkage to the hydroxyl group (substituting for hydrogen), linked directly to the polymer backbone by substituting for the hydroxyl group, or incorporated into an organic group which is linked to the polymer.

Attachment of non-protein organic or inorganic groups to the polymer modifies the hydrophilicity and the degradation rate and mechanism of the polymer. Protecting group chemistry may also be used to modify the hydrophilicity of the material. One skilled in the art will recognize that a wide variety of non-protein organic and inorganic groups may be added to or substituted for the hydroxyl groups in the polymer to modify its properties.

To further control or regulate interaction of the bone substitute composition with cells, a biomolecule component can be employed. The biomolecule component can be incorporated into the bone substitute composition using various conventional techniques. For example, the biomolecule can be coupled to the hydroxyl groups of the condensation polymer or integrated into the polymer backbone of the bone substitute composition or encapsulated within the condensation polymer. The biomolecule component can include growth factors which may be exploited to recruit cells to a bone defective site or promote specific metabolic or proliferative behavior in cells that are at the site or seeded within the bone substitute composition. Exemplary growth factors include, without limitation, TGF-β, acidic fibroblast growth factor, basic fibroblast growth factor, epidermal growth factor, IGF-I and II, vascular endothelial-derived growth factor, bone morphogenetic proteins, platelet-derived growth factor, heparin-binding growth factor, hematopoetic growth factor, peptide growth factor and, mixtures and combinations thereof.

In one embodiment, the biomolecule component includes a biological growth component, such as, but not limited to, biological signaling molecules and/or a growth compound. A non-limiting example of a suitable growth compound is BMP-2.

The bone substitute composition can be provided in the presence of a biomolecule component or in the absence of a biomolecule component. Further, the bone substitute composition can be provided in the presence of seeding with cells or in the absence of seeding with cells. The cells can include bone-forming cells, such as osteoprogenitor cells, the bone substitute composition can be colonized by loading or migrating the osteoprogenitor cells. The biomolecule component and/or the seeded cells can be effective to contribute to promoting the growth and formation of new bone. In alternate embodiments, the bone substitute composition is capable to serve as a scaffold for new bone growth to generate new bone in a bone defective site of a patient in the absence of a biomolecule component or in the absence of seeding with cells or in the absence of a biomolecule component and in the absence of seeding with cells.

The shape of the condensation polymer, e.g., poly(glycerol sebacate), can be manipulated for specific bone engineering applications. Non-limiting suitable shapes include, but are not limited to, particles, tubes, spheres, strands, coiled strands, films, sheets, fibers, meshes and combinations thereof. For example, the condensation polymer can be synthesized in the form of cylindrical tubes and sheets for long bone and flat bone engineering applications, respectively. The flat bone engineering applications include primarily the craniofacial area where distinguished and different trajectory forces, mainly from the masticatory apparatus, play a major role in the craniofacial bone remodeling, regeneration and trabecular orientation.

The bone substitute compositions can optionally include polymers in addition to the condensation polymer, e.g., poly(glycerol sebacate), formed by the reaction of glycerol and a diacid. The bone substitute compositions can include biodegradable and non-biodegradable polymers. One or more of these other polymers can be combined with the condensation polymer, e.g., poly(glycerol sebacate) in blends and adducts. Non-limiting examples of suitable biodegradable polymers can include, but are not limited to, natural polymers and their synthetic analogs, including polysaccharides, proteoglycans, glycosaminoglycans, collagen-GAG, collagen, fibrin, and other extracellular matrix components, such as elastin, fibronectin, vitronectin, and laminin. Suitable hydrolytically degradable polymers known in the art include, for example, polyesters, polyanhydrides, polyorthoesters, polyphosphazenes, and polyphosphoesters. Suitable biodegradable polymers known in the art, include, for example, polyhydroxyacids, polypropylfumerates, polycaprolactones, polyhydroxyalkanoates, poly(amide-enamines), polyamides, poly(amino acids), polyacetals, polyethers, biodegradable polycyanoacrylates, biodegradable polyurethanes and polysaccharides. Non-limiting examples of suitable biodegradable polymers include but are not limited to, polylysine, poly(lactic acid) (PLA), poly(glycolic acid) (PGA), copolymers and mixtures of PLA and PGA, e.g., poly(lactide-co-glycolide) (PLG), poly(caprolactone) (PCL), poly(lactide-co-caprolactone) (PLC), and poly(glycolide-co-caprolactone) (PGC). Those skilled in the art will recognize that this is an exemplary, not comprehensive, list of biodegradable polymers. The properties of these and other polymers and methods for preparing them are further described in the art.

Non-limiting examples of suitable non-biodegradable polymers can include but are not limited to polypyrrole, polyanilines, polythiophene, and derivatives thereof. Electrically conductive polymers may be useful to provide additional stimulation to seeded cells or neighboring tissue. Exemplary non-biodegradable polymers include, but are not limited to, polystyrene, polyesters, non-biodegradable polyurethanes, polyureas, poly(ethylene vinyl acetate), polypropylene, polymethacrylate, polyethylene, polycarbonates, poly(ethylene oxide) and, mixtures and combinations thereof.

Without intending to be bound by any particular theory, it is believed that the elastomeric and polymeric properties of the bone substitute compositions provide the ability to transmit the extracellular/environmental mechanical influences, found to be as important as soluble and adhesive factors that bind to cell-surface receptors, to the differentiating progenitor cells. Further, it is believed that the elasticity of the condensation polymer, e.g., poly(glycerol sebacate, allows the bone substitute compositions to overcome the brittleness of conventional ceramic materials and offer the advantage of mechanical stimulation without compromising biocompatibility, resorbability and porosity structure (e.g., size and interconnectivity) beneficial for bone regeneration.

### Examples

Scaffolds were synthesized using a bone substitute composition in accordance with an embodiment of the invention. The scaffolds included poly(glycerol sebacate) synthesized in the form of tubes for a long bone defective, e.g., orthopedic, and in the form of sheets for a flat bone defective, e.g., craniofacial. The PGS scaffolds were synthesized with and without hydroxyapatite (HA) particles. The presence of HA enhances the osteoconductivity of the scaffold. The synthesized PGS scaffolds were characterized in vitro based on their mechanical properties and support of osteoprogenitor cells' viability, adhesion, proliferation and differentiation. The synthesized PGS scaffolds were characterized in vivo for their ability to regenerate a critical size defective in a rabbit ulna model.

### 1. Synthesis of Poly(Glycerol Sebacate) (PGS)

Poly(glycerol sebacate) (PGS) was synthesized by polycondensation of 0.1 mole each of glycerol and sebacic acid (i.e., a 1:1 ratio of glycerol and sebacid acid) at a temperature of 120° C. under argon for 24 hours prior to reducing the pressure from 1 Torr to 40 mTorr over 5 hours. The reaction mixture was kept at 40 mTorr and 120° C. for 48 hours. The yield was a transparent, almost colorless elastomer that did not swell or dissolve in water.

a. PGS Tubes

A portion of the PGS yield was shaped into tubes. The PGS tubes were synthesized with 75-150 μm porosity and at 24 hours curing time. Fabrication was carried out in a glass mold (OD=8.9 mm, ID=6.8 mm, length=75 mm) centered by a stainless steel rod enveloped by a heat-shrinkable (“HS”) tubing (OD=5.28 mm, ID=4.75 mm) (Insultab, Woburn, Mass.). The glass mold was wiped and coated with a 1% w/v hyaluronic acid (53747, Fluka) solution to form a substantially even layer that was left to dry for 24 hours at 37° C. in a vacuum. Porogen particles (NaCl, EMD Chemicals, Gibbstown, N.J.) having a particle size from 75-150 μm were compactly packed into the glass mold and the salt-packed mold was placed in a hybridization incubator at greater than 80% relative humidity for salt fusion (to achieve proper pore interconnectivity in the synthesized PGS scaffold). Then, it was placed in a vacuum oven to dry overnight at a pressure of 100 mTorr and a temperature of 37° C. The packed salt was wetted by a solution of 20% PGS in tetrahydrofuran and curing was carried out in the vacuum oven for 24 hours at a pressure of 100 mTorr and a temperature of 150° C. To release the cured PGS from the glass mold, the hyaluronic acid was dissolved in a water bath for 1 hour and the scaffold was then gently pushed out. The sleeve covering the stainless steel rod was allowed to shrink in the oven and was gently withdrawn then the rod, and the scaffold was transferred into a water bath with stirrer for 3 days with changing water to leach out the salt particles. The scaffold was then transferred into 15 mL centrifuge tube filled with de-ionized water and placed in a dry-ice box for 1 hour to freeze after which it was lyophilized for 3 days. Scaffolds were stored in the desiccators until used.

b. PGS Sheets

A portion of the PGS condensation polymer was formed into sheets. The sheets were synthesized in molds in the form of metallic rings attaching by magnet to a metallic base. A single ring was 1 mm high and the sheets were synthesized in 2 mm thickness (e.g., two rings)

c. PGS with HA Particles (Referred to as PGS 20% HA)

For the tubes and sheets, HA particles (<200 nm, Sigma) were thoroughly mixed with the porogen (NaCl, EMD Chemicals, Gibbstown, N.J.) in a proportion of 50 mg for each 4 mg of salt. As each 4 mg of salt was wetted by 250 mg of PGS monomer, the HA was considered to be 20% of PGS in the final synthesized scaffold (50 mg HA for each 250 mg PGS).

A portion of the PGS sheets were synthesized in a hybrid manner such that the first ring received the porogen powder and the overlaying second ring received porogen mixed with HA (referred to as PGS 20% HA).

d. PGS Tubes Soaked in a Calcium Phosphate (CaP) Solution

A mineral deposit was deposited on the surface of a PGS scaffold. A calcium phosphate (CaP) solution was prepared by combining a mix of 2:3 CaCl₂ solution 0.4M and a phosphate buffer solution 1.91 mM (Na₃PO₄12H₂O 0.015M, NaCl 2.8M, KCl 0.1M, Dextrose 0.12M and Hepes 0.5M, all in equal proportions then diluted 1:1 with double distilled water). The PGS condensation polymer was dipped into the CaP solution for about 20 minutes.

### 2. PGS Mechanical Properties

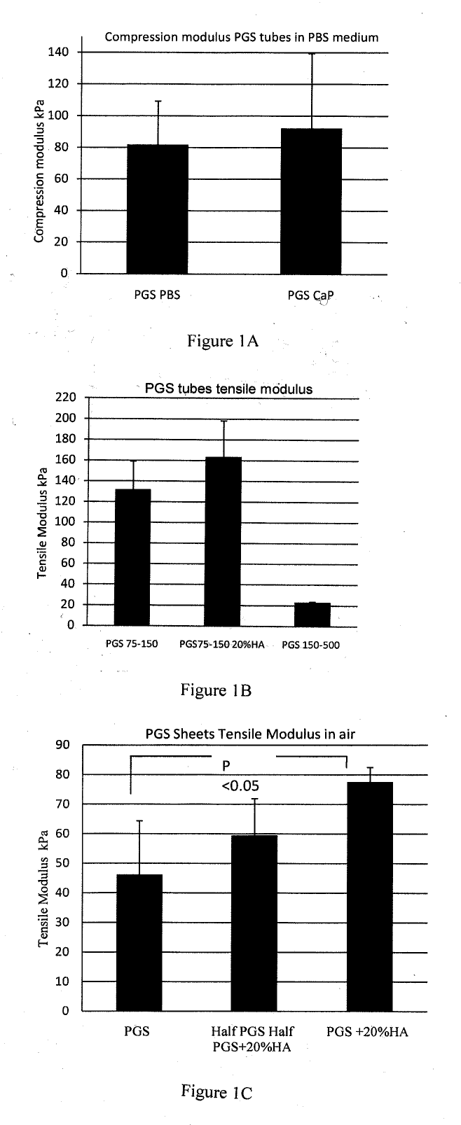

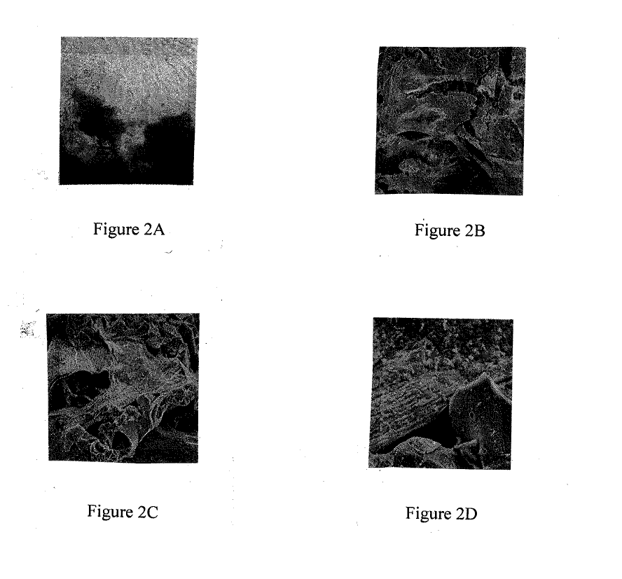

The tensile and compressive moduli for the PGS tubes and sheets were tested. The results are shown in FIGS. 1A, 1B and 1C. For the tensile test, the tubes were cut into segments of 5 mm in length that was hooked apart at a rate of 0.1 mm/second until failure occurred. The compressive test took place in a PBS environment where the 5 mm segment was subjected to a preload of 0.05 N and 10 times 10% compression per minute. The compression modulus of the synthesized PGS tubes with and without the CaP soak is shown in FIG. 2A. It is demonstrated that the CaP soak results in an increased compression modulus but does not significantly change the compression modulus of the PGS tubes. The tensile modulus of synthesized PGS tubes with and without HA is shown in FIG. 2B. It is demonstrated that the presence of HA does not significantly increase the tensile strength in the synthesized PGS tubes. The tensile modulus in air of synthesized PGS sheets with and without HA is shown in FIG. 2C. It is demonstrated that the presence of HA significantly increases the tensile strength of the synthesized PGS sheets.

### 3. Cells Seeding and In Vitro Assessment of Cells Viability, Adhesion, Proliferation and Differentiation on PGS Scaffolds

Scaffolds were constructed using the synthesized PGS sheets. The scaffolds were seeded either with human or rabbit bone marrow mesenchymal stromal cells (h or rBMSC). Cell culture took place in AMEM media with 20% FBS supplemented with 2 mM glutamine, 100 U/ml penicillin, 100 μg/ml streptomycin. The synthetized PGS sheets were cut into 3.5×3.5 mm² sheets, sterilized and loaded with 2×10⁵ cells in 150 of culture media.

a. PGS Sterilization

The day before utilization or cell seeding, the PGS scaffolds were autoclaved at 120° C. for 30 minutes and then soaked in a series of ethanol 70%, 50% and 25% each for 30 minutes, followed by PBS, 2 times for 30 minutes each, and then culture media overnight at a temperature of 37° C. and 5% CO₂.

b. Cell Viability

The viability of cells on PGS was investigated by simple replating into an empty culture dish. FIG. 2A is a SEM showing viable cells migrating out of a replated PGS scaffold at 20×.

c. Cell Adhesion

The adhesion of cells on PGS was assessed by SEM. FIG. 2B is a SEM showing confluent BMSC on the surface of a PGS scaffold at 1000×. FIG. 2C is a SEM showing BMSC attached to the PGS scaffold at 2000×. FIG. 2D is a higher magnification SEM of the SEM in FIG. 2C to emphasize the close integrity of a cell to the PGS scaffold at 10000×.

d. Cell Proliferation

MTT assay was employed to assess cell proliferation. This assay was based on the ability of themitochondrial dehydrogenases to oxidize thiazolyl blue a tetrazolium salt [3-(4,5-dimethylthiazole-2-yl)-2,5-diphenylterazolium bromide] to an insoluble blue formazan product. The absorbance readings were performed on an ELISA plate reader using test and reference wavelengths of 570 and 670 nm, respectively.

e. Cell Differentiation

PGS scaffolds were assessed for cell differentiation. The assessment was conducted by the detection of mineral deposits on the scaffolds for a culture period of 28 days by Alizarin Red S and Von Kossa Stains as well as detection of osteogenic markers (Runx2, Col1, OC, OPN, BSP) by immunohistochemistry.

4. PGS Scaffold Implantation into Critical Size Induced Rabbit Ulna Defective

PGS tubes were dipped in PBS for 20 minutes and other PGS tubes were dipped in CaP for 20 minutes. Each of the tubes were inserted into an induced 1.6 cm rabbit ulna defective. No cells were loaded on these scaffolds.

a. Rabbit Subjects

Four, 16 week-old white New Zealand rabbits (weight=3.5 kg±0.01 kg) were obtained. Two of the rabbits, identified as R4393 and R4394, received calcium phosphate-soaked PGS. The other two rabbits, identified as R4395 and R4396, received PBS-soaked PGS. Rabbits R4393 and R4395 were sacrificed after 4 weeks. Rabbits R4394 and R4396 were sacrificed after 8 weeks.

b. Surgical Procedure

The rabbits were anesthetized by IM Ketamine (80-100 mg/kg)-Xylazine (5-10 mg/kg) injection and the left forelimbs were shaved, disinfected and the surgical site was cleanly isolated. Access to the left ulna was made through cutaneous and subcutaneous incisions. The muscular fascia was bluntly dissected until reaching the surgical site. The ulna was clearly exposed and the length of the segment to be removed was marked by a sterile gauge previously opened on the exact scaffold length. The marked segment was cut proximally and distally by rotary fissure bur. The previously CaP- and PBS-soaked PGS tubes were inserted in the created surgical defective. The wound was closed by subcutaneous, and then by cutaneous, interrupted sutures. Two of the rabbits were sacrificed at 4 weeks and two of the rabbits were sacrificed at 8 weeks to assess early bone formation and remodeling.

c. Assessment

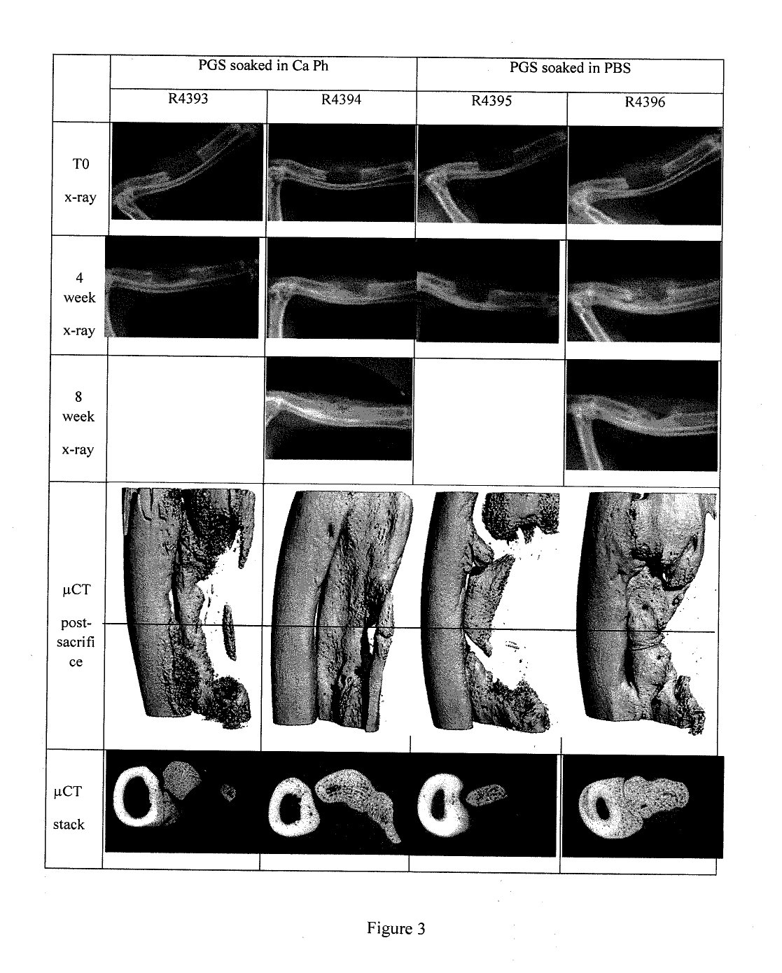

Radiographs were performed on the day of surgery and 4 weeks and 8 weeks post-surgery as shown in FIG. 3. At sacrifice, the operated ulna and radius were retrieved, fixed in paraformaldehyde 3.7% for 24 hours and prepared for μCT and histological processing. The μCT was performed at 35 μm resolution, integration time of 299 msec, 55 keV, 142 μA with cone beam and continuous rotation. The scanned area was 22 mm in length (16 mm defective, 3 mm from each side). All μCT samples showed osteogenic activity out of the osteotomy ends or at the radius surface which points to the periosteum as the source of osteoprogenitor cells. Fusion of ulnar and radial bone was observed. The degree of bone remodeling varied between the specimens. Some showed compact newly formed bone, while in other regenerated defectives bone marrow trabeculae was apparent.

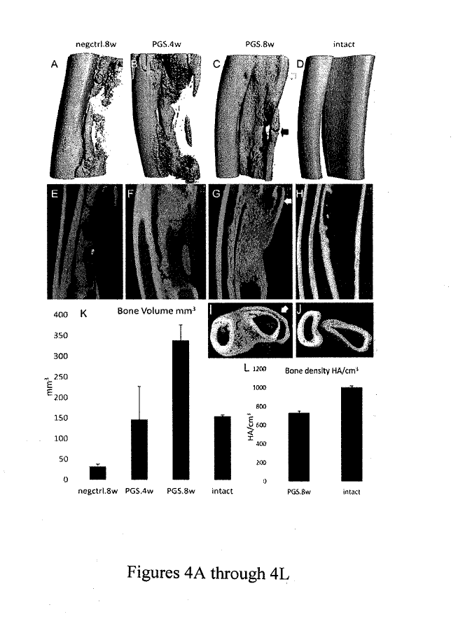

Further, bone regeneration was seen in both 4 and 8 weeks samples. Islands of new bone were observed in the 4-week PGS sample (PGS.4 w) as shown in FIG. 4B and in the 8-week negative control sample (negctrl.8 w) as shown in FIG. 4A. The 8-week PGS sample (PGS.8 w) exhibited complete bridging as shown in FIG. 4C. Based on observations of the pattern of bone deposition at 4 weeks, it appears that the bone deposition started from the periosteum at the ulna extremities of the defect, the periosteum of the radius leading to partial fusion and also from the migrating cells within the scaffold apparent from bone islands within the defect. The characteristic of the regenerated bone until 8 weeks was globally trabecular bone as shown in FIGS. 4C and 4G. However, cortical remodeling was observed as a second cortical layer concentric with the original cortical bone at the extremity of the ulna defect as shown (by the arrows) in FIGS. 4G and 4I. The bone deposition from migrating cells from proximal and distal sides of the defect may have resulted in a central non-union observed at 8 weeks as shown (by the arrow) in FIG. 4C.

Bone volume calculation is shown in FIG. 4K. It is demonstrated that there was a significantly higher amount of bone at 8 weeks (i.e., 338+/−38) as compared to the negative control (32.5+/−5.5). The intact ulna (intact) measured less bone volume as the marrow occupied entirely the shaft without cancellous trabeculae (see FIG. 4H) while the regenerating bone within the defect was trabecular in nature and expected to remodel to compact bone with time. Bone density calculated by hydroxyapatite particles/cc (see FIG. 4L) confirmed the overall higher density of the intact ulna bone compact in nature (1004+/−8) as compared to the lower density trabecular regenerated bone after 8 weeks (733+/−11).

Further, immunohistochemistry (IHC) analysis from periostin antibody revealed an abundance of periostin positivity at 4 weeks within and around the regenerating bone (immature) and the cartilage canals, also regarded as endosteum, pointing to the periosteal cells as main contributors to regeneration. The expression of periostin coincided with the abundance of hypertrophic chondrocytes indicating the endochondral pathway of ossification. Chondrocytes within the cartilage canals, the primary site of bone maturation, were periostin positive while the periostin expression was lost in osteocytes within mature bone. With bone maturation, at 8 weeks, the periostin expression was reduced being limited to the margins of bone and the cartilage phase (which was observed at 4 weeks) was absent.

### 5. Summary of Results

The tensile and compressive strengths of the synthesized PGS tubes were 0.14±0.04 MPa and 0.044±0.003 MPa, respectively, which was lower than the cortical or cancellous bone mechanical properties (cortical bone comp. st. 100-200 MPa, tensile st. 50-150 MPa, Young mod. 7-30 GPa, cancellous bone compressive strength 2-12 MPa, tensile st. 10-20 MPa, Young mod. 0.5-0.05 GPa) and still lower than the compressive strength of the conventional (non-coated, non-treated) HA/β-TCP materials (0.05-1.2 MPa) The elasticity of the material was close to a “bone friendly” elastic modulus (25-40 kPa) that favors cells differentiation into the osteogenic lineage. The results showed that the synthesized PGS material qualifies for a bone engineering scaffold satisfying its mechanical and biological requirements. The elastic properties of PGS are close to the elasticity considered to support osteogenic lineage differentiation, hence favoring bone regeneration. The PGS material supported BMSCs attachment indicating its favorable milieu for cellular function and upon in-vivo implantation. It was mechanically stable and able to recruit peripheral osteoprogenitor cells to migrate and colonize the scaffold-occupying gap synthesizing neo-bone.

Without intending to be bound by any particular theory, it is believed that the particular mechanical/elastic properties of the condensation polymer, PGS, permitted load transmission to the migrating cellular components allowing their differentiation and deposition of a mineralized matrix that completely bridged the ulna defective by newly regenerated bone in a period as short as 8 weeks (as shown in FIG. 3).

Other embodiments of the invention will be apparent to those skilled in the art from a consideration of the specification or practice of the invention disclosed herein. It is intended that the specification and examples be considered as exemplary only.

We claim:

1. An elastomeric, polymeric bone substitute composition capable to regenerate bone in the absence of a biomolecule component.

2. The bone substitute composition of claim 1, wherein the composition comprises a condensation polymer formed by a reaction of glycerol and diacid.

3. The bone substitute composition of claim 1, wherein the diacid is sebacic acid.

4. The bone substitute composition of claim 1, wherein the composition comprises poly(glycerol sebacate).

5. The bone substitute composition of claim 1, wherein the biomolecule component comprises a growth compound.

6. The bone substitute composition of claim 1, wherein the composition is seeded with bone-forming cells.

7. The bone substitute composition of claim 5, wherein the bone-forming cells are osteoprogenitor cells.

8. The bone substitute composition of claim 1, wherein the composition is effective to regenerate bone in vitro.

9. The bone substitute composition of claim 1, wherein the composition is effective to regenerate bone in vivo in a bone defective site of a patient.

10. The bone substitute composition of claim 9, wherein the composition is effective to regenerate bone in a period of 8 weeks or less.

11. The bone substitute composition of claim 1, wherein the composition is effective to regenerate bone in the absence of seeding with cells.

12. The bone substitute composition of claim 1, further comprising a porogen.

13. The bone substitute composition of claim 1, further comprising a polymer selected from the group consisting of biodegradable polymers, non-biodegradable polymers and mixtures thereof.

14. The bone substitute composition of claim 1, wherein the composition is in a shape selected from the group consisting of particles, tubes, spheres, strands, coiled strands, films, sheets, fibers, meshes and combinations thereof.

15. The bone substitute composition of claim 1, wherein the composition is used in a bone defective site in an area of a patient, the area selected from the group consisting of orthopedic, dental and caraniofacial areas.

16. The bone substitute composition of claim 4, wherein the poly(glycerol sebacate) is capable to regenerate bone in a bone defective site of a patient.

17. A method of preparing a bone substitute composition comprising reacting glycerol and diacid to form a condensation polymer, in the absence of a biomolecule component.

18. A method for regenerating bone in a bone defective site of a patient, comprising:
preparing a bone substitute composition by reacting glycerol and diacid to form a condensation polymer; and
implanting the bone substitute composition, in the absence of a biomolecule component, in a bone defective site of a patient.

19. A method for regenerating bone, comprising:
preparing a bone substitute composition including a condensation polymer which is a reaction product of glycerol and diacid, wherein the bone substitute composition is effective to regenerate bone in a period of 8 weeks or less.

20. The method of claim 19, wherein the bone substitute composition does not comprise a biomolecule component.
